(19) United States
(12) Patent Application Publication
(10) Pub. No.: US 2009/0204005 A1
(43) Pub. Date: Aug. 13, 2009

(54) PUNCTURE RESISTANT CATHETER FOR SENSING VESSELS AND FOR CREATING PASSAGES IN TISSUE

(75) Inventors: Thomas M. KEAST, Sunnyvale, CA (US); Eric J. GWERDER, Fremont, CA (US); Aaron M. WEISS, San Francisco, CA (US)

Correspondence Address: LEVINE BAGADE HAN LLP, 2400 GENG ROAD, SUITE 120, PALO ALTO, CA, 94303, US

(73) Assignee: Broncus Technologies, Inc., Mountain View, CA (US)

(21) Appl. No.: 12/027,795

(22) Filed: Feb. 7, 2008

(51) Int. Cl. A61B 8/14; A61M 25/06

(52) U.S. Cl. 600/461; 604/164.01; 604/170.03

(57) ABSTRACT

Devices and methods are disclosed for providing a sensing element to scan tissue and detect the presence of structures within the tissue to avoid the structures when performing a procedure on the tissue. A tissue penetrating member or needle is extendable from the device to create a channel or opening through the wall of an airway. The device is sufficiently flexible to navigate turns of at least 90 degrees. The distal section of the device includes puncture resistant sections, a curved needle tip, and depth limiting features that serve to create a channel in the lumen wall without causing collateral damage to the instruments, or patient.

### FIELD OF THE INVENTION

The invention is directed to devices for sensing movement within tissue at a target site to scan for the presence or absence of structures such as blood vessels so that a procedure may be performed at the target site in a safe manner.

### BACKGROUND OF THE INVENTION

When performing procedures through an endoscope, bronchoscope, or other such device there is a risk that the procedure might disrupt structures beneath a tissue surface (such as blood vessels), where the disruption then causes significant complications.

One such area is within the airways of the lungs where puncturing of a blood vessel beneath the airway surface can result in significant bleeding. In cases where a scope type device is used, the bleeding obstructs the ability of the medical practitioner to visualize the damaged area resulting in an escalation of complications. In some cases, a patient's chest must be opened to stem the bleeding.

Such risks occur in many types of scope-based procedures, including but not limited to lung based approaches. For example, creation of collateral channels in COPD patients poses such risks. For example see U.S. Pat. No. 6,692,494; U.S. patent application Ser. Nos. 09/947,144, 09/946,706, and 09/947,126 all filed on Sep. 4, 2001; U.S. Patent Application No. filed on Sep. 4, 2002: U.S. patent application Ser. No. 11/335,263, filed on Jan. 18, 2006; U.S. patent application Ser. No. 11/562,947, filed on Nov. 22, 2006; each of which is incorporated by reference herein in its entirety. In addition, biopsy procedures, and transbronchial aspiration procedures arc a few procedures that present the same risk of penetrating a blood vessel within the lungs.

The problem is further compounded when accounting for motion of the tissue. For example, because airway or other lung tissue moves due to tidal motion of the lungs (as a result of the mechanics of breathing), it is difficult to visually identify an area that was previously scanned unless the scanning device remains relatively stationary against the tissue. Moreover, the difficulty increases when considering that the procedure takes place through the camera of a bronchoscope or endoscope.

Aside from the risk to the patient, once the medical practitioner punctures a blood vessel, that practitioner is often understandably hesitant or risk averse when performing future procedures. As a result, while the benefit of these procedures is well known, the risks of complications may reduce the overall success of the procedure.

Additionally, during bronchoscope surgeries it is not uncommon for the interventionalist or surgeon to navigate the bronchoscope through 90 to 180 degree bends. For example, a bend of this nature may be required to reach an upper lobe of a lung in an airway bypass procedure as described in U.S. Pat. No. 6,692.494 and incorporated herein by reference in its entirety. Likewise, the catheter being advanced through the bronchoscope must also be able to bend to the same degree as the bronchoscope. The catheter must be able to bend and maintain its functionality. In the case of a catheter carrying an extendable needle, and being urged through a working channel of a bronchoscope, a number of challenges may arise including, for example, increased friction between the needle and the catheter's working lumen. The needle may engage or puncture the working lumen as it is advanced through a sharp turn. The needle may also damage the bronchoscope, or worse, injure the patient. In view of the above, a need remains to increase the operation and safety of catheter systems through turns of up to 180 degrees. Such a need remains in procedures that create channels to vent trapped gasses within the lungs, transbronchial aspiration procedures, transesophageal procedures, biopsy procedures, use of cytology brushes, etc. Furthermore, the need may arise in any lung based procedure or other procedures in other parts of the body.

### SUMMARY OF THE INVENTION

The invention relates to devices and methods for sensing structures within tissue (such as blood vessels or other organs) while performing a procedure at the site.

The catheter member can be a tubular member. The catheter member can be a polymeric tube or a reinforced polymeric tube. As described herein, it may have one or more lumens to accommodate the variations of the devices within this disclosure.

The sensing assembly is used to scan the tissue to minimize causing undesirable injury to the patient. As discussed below, any number of sensing modes may be incorporated into the device. In one variation of the invention, a Doppler ultrasound transducer assembly is incorporated for sensing blood vessels within tissue.

In variations of the device, the sensing assembly is offset from an axis of the catheter assembly. The sensing assembly may also be positioned distal to the main catheter lumen. Doing so improves the ability of the sensing assembly to contact tissue surfaces when the device is advanced along body conduits. In addition, this offset and/or extension feature improves the ability to see the tip of the sensing assembly when the device is used with a scope type device.

The invention further includes methods of treating tissue, where the method includes selecting an area in the tissue for treatment, advancing the device into the lung to a tissue site, where the device includes a sensing assembly affixed to a catheter to sense for the presence or absence of blood vessels. The device may then allow for the use of a needle member comprising a curved needle that creates a passage. The device also includes various depth limiting features, such as a transition surface that causes tactile or sensory feedback to a physician if the device is advanced too far out the distal end of the sensing assembly, which can act as a stop. In addition, the needle assembly can include visual indicators to allow a physician to observe proper advancement of the needle.

As noted herein, one variation of the device permits scanning the tissue site by placing the sensing assembly in contact with the tissue site. However, various sensing assemblies may permit non-contact scanning. Regardless of whether the sensing tip contacts the tissue, creation of the opening or passage may be performed without significant movement of the scanning assembly. Such a benefit is apparent as medical practitioners may lose track of the scanned tissue if they are required to substitute or move the scanning assembly to create an opening.

The invention includes a medical device for sensing structures beneath tissue and penetrating tissue, the device comprising an elongate sheath having a near portion, a far portion, and a handle located on the near portion, the elongate sheath including at least one lumen extending through the handle to the far portion and exiting at a sheath opening, the elongate sheath being sufficiently flexible to navigate through a working lumen of a bronchoscope as well as a tortuous anatomy, a sensing element spaced distally from the sheath opening and positioned such that an active surface of the sensing element is able to scan in a distal direction to inspect tissue distal to a front tip of the elongate sheath while leaving the sheath opening proximally spaced from tissue, where the sensing element is coupleable to a sensing monitor, an elongate body member having a hub located at a proximal end, and a needle tip located at a distal end, the body member and needle tip being slidably located through the sheath lumen, where a length of the body member is greater than a length of the elongate sheath such that when the hub advances against the handle, the needle tip extends distally to the sensing element.

In another variation, the invention includes a medical device for sensing structures beneath tissue and penetrating tissue at a remote site in tortuous anatomy, the device comprising an elongate sheath having a near portion, a far portion, and a handle located on the near portion, the elongate sheath including at least one lumen extending through the handle to the far portion and exiting at a sheath opening, the elongate sheath having a reinforcing member extending at least partially therethrough and being sufficiently flexible and long to navigate through tortuous anatomy, a sensing element being coupleable to a sensing monitor, an elongate body member having a hub located at a proximal end, and a needle tip located at a distal end, the body member and needle tip being slidably located through the sheath lumen, where a length of the body member is greater than a length of the elongate sheath such that when the hub advances against the handle, the needle tip extends distally from the sheath opening, where the elongate body member has a column strength to allow the needle tip to puncture tissue when the hub advances against the handle, and where the needle tip comprises a curved tip and where a front portion of the curved tip curves towards a centerline of the needle tip thereby minimizing the front portion of the curved tip from interfering with an interior wall of the sheath lumen.

The invention also includes methods of creating an opening in tissue located in tortuous anatomy at a preferred site. In one variation the method includes advancing a device through the tortuous anatomy. The device includes a sensing element and a sheath where the sensing element extends distally beyond the sheath opening, scanning the tissue with the sensing element to identify the preferred site. The sensing element is pressed against the preferred site while keeping the sheath Opening spaced from the preferred site. The needle tip and elongate body member is extended from the sheath opening, where a transition section between the needle tip and the elongate body member comprises a reduced diameter section distal to an increased diameter section. In another variation, the elongate member comprises an increased diameter section. The needle tip pierces the tissue such that the transition section interferes with tissue causing sensory feedback at a proximal end of the elongate member. Elastic tissue will also constrict and conform to the reduced section such that continued advancement will result in an interference between the tissue and needle shaft.

### BRIEF DESCRIPTION OF THE DRAWINGS

FIG. 1 shows a variation of a system as described herein.

FIG. 2A shows a far end of a sheath according to the present invention.

FIG. 2B shows a hub portion advanced against the handle of the sheath to advance a working device, in this case a needle assembly, out of the sheath.

FIG. 2C shows a magnified view of the far end of a sheath with a needle assembly advanced from a sheath opening.

FIG. 3A shows a view of a distal end of a needle assembly for use with the present system.

FIG. 3B shows a curved needle tip.

FIG. 3C shows proximal and distal ends of a needle assembly.

FIGS. 4A-4F show additional variations of the ends of sheath to offset and extend a sensing element from the main lumen.

FIG. 5A illustrates an example of using the device to scan a target site prior to performing a procedure at the target site.

FIG. 5B shows an example of a needle tip penetrating an airway wall and use of a depth limiting feature to prevent excessive advancement of the device.

### DETAILED DESCRIPTION OF THE INVENTION

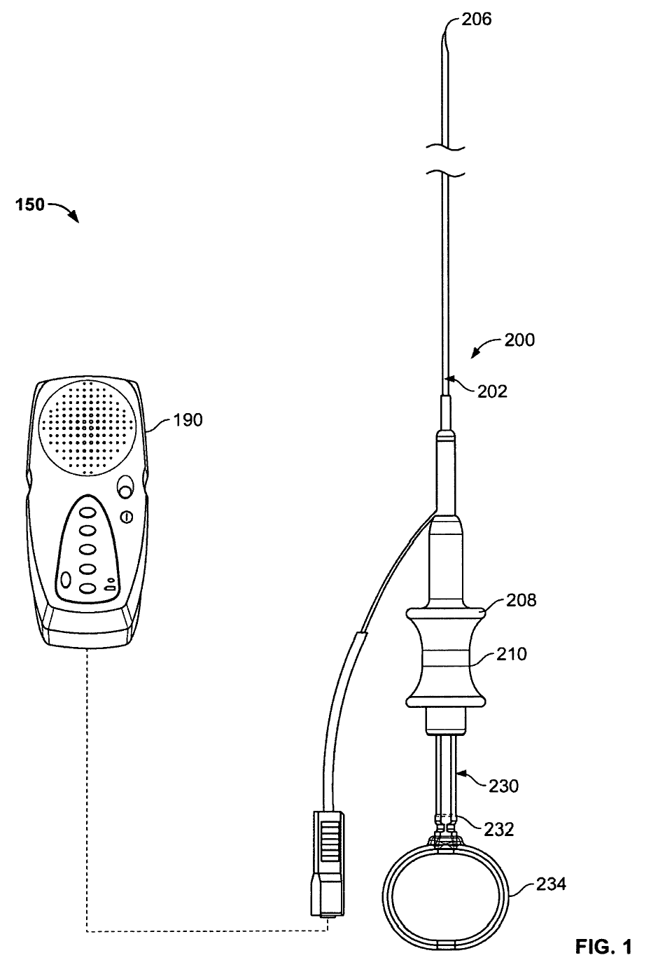

FIG. 1 illustrates a view of a variation of an inventive system 150 incorporating various features as described herein. The system generally includes a device 200 having an elongate sheath 202 with a sensing element 206 at a far end of the sheath 202. The far end of the sheath 202 shall be atraumatic so that movement of the sensing element 206 across tissue does not cause damage to the tissue. The elongate sheath 202 includes a working device extending therethrough. In this variation, the working device comprises a needle assembly 230. The device 200 is also coupled to (or connectable to) a control system 190 that is configured to assist the physician in scanning the tissue site. In the present variation, the control system 190 assists the physician in detecting whether blood vessels are at or near a particular target site. The control system 190 may be any type of unit that confirms the presence or absence of blood vessels. As such, it may be a thermal based system (incorporating a temperature detecting element, thermocouple, RTD, etc.), light based system (incorporating a fiber-optic system to measure reflected light or energy), ultrasound based system, or Doppler ultrasound based system. The sensing element may provide an image or may simply provide sound or other data. The system 150 may also include various other components as required (e.g., fluid sources, medications, vacuum sources for aspiration, etc.)

As will be discussed below, a variation of the invention includes a Doppler ultrasound based sensing element 206 and control system 190. However, other modes are within the scope of this invention and regardless of the mode incorporated by the sensing assembly, the system 150 may include a user interface that allows the user to determine the presence or absence of a blood vessel at the target site. Typically, the user interface provides an audible confirmation signal. However, the confirmation signal may be manifested in a variety of ways (e.g., light, graphically via a monitor/computer, etc.)

Although depicted as being external to the device, it is contemplated that the control system 190 may alternatively be incorporated into the device 200. Moreover, the system 150 may incorporate any number of connectors or fittings that allow for either permanent or detachable connections of the fluid source, control system and/or any other auxiliary systems used with the system 150.

When using Doppler ultrasound to detect the presence of blood vessels within tissue, the ultrasound can operate at any frequency in the ultrasound range but preferably between 2 Mhz-30 Mhz. It is generally known that higher frequencies provide better resolution while lower frequencies offer better penetration of tissue. In one embodiment of the present invention, because location of blood vessels does not require actual imaging, there maybe a balance obtained between the need for resolution and for penetration of tissue. Accordingly, an intermediate frequency may be used (e.g., around 8 Mhz). In another embodiment of the invention, it is desirable to operate at a frequency of about 30 Mhz. A variation of the invention may include inserting a fluid or gel into the airway to provide a medium for the Doppler sensors to couple to the tissue to detect blood vessels. In those cases where fluid is not inserted, the device may use mucus found within the airway to directly couple the sensor to the wall of the airway.

As noted above, Doppler ultrasound was found to be an efficient way to identify blood vessels. As such, the control system 190 can be configured to communicate with an analyzing device or control unit adapted to recognize the reflected signal or measure the Doppler shift between the signals. The source signal may be reflected by changes in density between tissues. In such a case, the reflected signal will have the same frequency as the transmitted signal. When the source signal is reflected from blood moving within a vessel, the reflected signal has a different frequency than that of the source signal. This Doppler Effect permits determination of the presence or absence of a blood vessel within tissue. The Doppler system described herein comprises a Doppler ultrasound mode of detection. However, additional variations include transducer assemblies that allows for the observation of the Doppler Effect via light or sound as well.

Turning now to the device 200, the elongate sheath 202 typically has a sufficient length that allows a physician to advance the sheath 202 through tortuous anatomy to a remote site. As noted above, such a device is useful in the lungs, vasculature, or other such tortuous anatomy. Accordingly, variations of the sheath 202 are fabricated with sufficient flexibility and column strength to reach the intended target site. Although various sizes are within the scope of the invention, one configuration includes a sheath diameter of 1.8 mm with a 21 GA needle having a penetration depth of 10.9 mm. In one embodiment, the working length of the device can range from 1320 to 1473 mm.

As shown in FIG. 1, the sheath 202 also includes a handle portion 208 at a near end of the sheath. Typically, the handle portion 208 may be of any known handle type configuration commonly used with medical devices. However, in the illustrated variation, the handle portion includes one or more grooves 210. The groove 210 allows for a physician to grasp the handle and actuate the working device or needle assembly 230 with a single hand. The sheath 202 includes a lumen extending from the handle portion 208 through the far end. As shown, the working device can be inserted into the lumen via the handle portion 208. In the illustration shown, the working device includes a hub 232 with a ring member 234 to assist in single handed operation. Clearly, any number of features can be incorporated into the handle 208 and hub 230 to improve the ability of the physician to manipulate/actuate the device.

Although the variations discussed below include a needle assembly for the working device, the working device can include any number of devices. For example, the working device can include aspiration needles, transbronchial aspiration needles, biopsy devices, brushes, forceps, and other such devices that are able to be advanced through the lumen of the sheath 202 to the target site at the end of the device 200. In most variations, such medical appliances are of a small diameter. The use of the handle 208 provides a more convenient opening for insertion of a small device. For instance, attempting to insert an aspiration needle (e.g., 17-21 GA) into a small diameter lumen will be a lime consuming effort. Use of the hub 208 permits the medical practitioner to rapidly insert such a small sized appliance into the device 200.

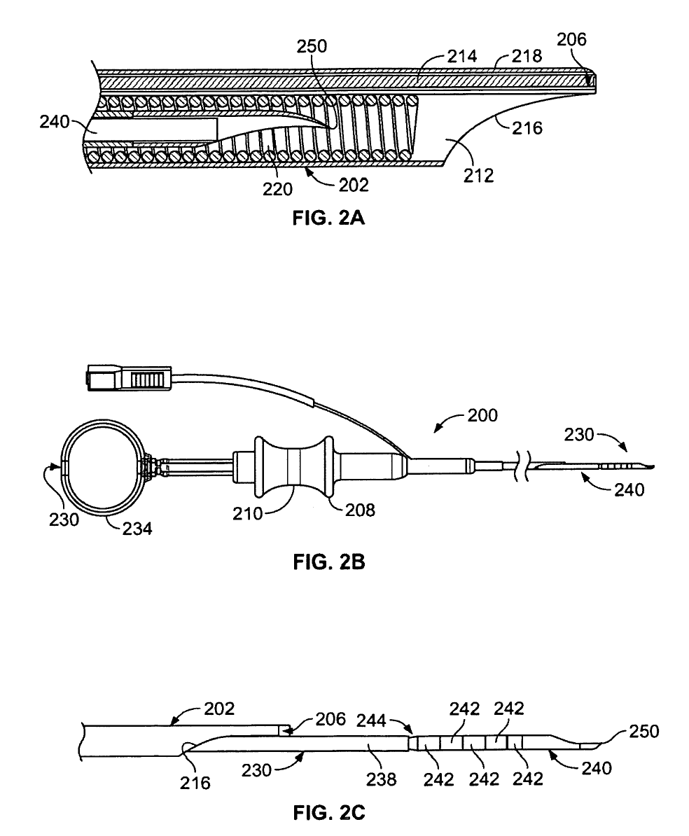

FIG. 2A illustrates a partial cross sectional view of the device 200 of FIG. 1. In this variation, the sheath 202 is a multi-lumen tube. As noted above, the first main lumen 212 extends through the sheath 202 and exits the near end of the sheath at the handle (not shown). The main lumen 212 exits the far end of the sheath 202 through a sheath opening 216. The exit 216 has a curved, or tapered, opening. The second lumen 214 extends parallel to the main lumen 212 and contains a sensing clement 206. The sensing element is offset, and distal to the sheath 202 or exit 216. Although the illustrated variation is shown as having two lumens, the present invention contemplates a device having any number of lumens. For example, the device may include a single lumen that contains both the sensing element and the working device. Alternatively, one or more lumens can be used to deliver fluids, apply suction, or provide illumination or visualization to the target site.

Turning now to the sensing element 206, in the illustrated variation the sensing clement 206 is situated such that an active surface of the sensing element is able to scan in a distal direction to inspect tissue distal to a front tip of the elongate sheath. Such a configuration permits the sensing element 206 to scan the target site for structures beneath the tissue prior to insertion of the working device into the target site. Clearly, variations of the invention can include sensing elements that are angled or scan in a radial or an oblique direction relative to the sheath 202.

The sensing element can be any modality that is capable to scan beneath a surface of tissue. For exemplary purposes, the control system 190 and sensing element 206 are discussed herein as being a Doppler ultrasound system. As such, the sensing element 206 includes the sensing tip that is coupled to the power supply 190 as is known by those familiar with such systems. For example, the sensing element 206 may include any number of conducting members (e.g., wires) extending along the sheath 202 (either internally or externally to the sheath 202). In any case, these conducting members provide the energy and controls for the sensing element 206. In the case of Doppler ultrasound, the conducting members couple an ultrasound source 190 to the sensing element 206 where the element comprises an ultrasound transducer assembly or lens. The sensing clement 206 can be covered by a layer or coating to ensure biocompatibility and durability and an atraumatic tissue contact interface. The transducer or transducers may comprise a piezo-ceramic crystal (e.g., a Motorola PZT 3203 HD ceramic). In the current invention, a single-crystal piezo (SCP) is preferred, but the invention does not exclude the use of other types of ferroelectric material such as poly-crystalline ceramic piezos, polymer piezos, or polymer composites. The substrate, typically made from piezoelectric single crystals (SCP) or ceramics such as PZT, PLZT, PMN, PMN-PT; also, the crystal may be a multi layer composite of a ceramic piezoelectric material. Piezoelectric polymers such as PVDF may also be used. Micromachined transducers, such as those constructed on the surface of a silicon wafer are also contemplated. As described herein, the transducer or transducers used may be ceramic pieces coated with a conductive coating, such as gold. Other conductive coatings include sputtered metal., metals, or alloys, such as a member of the Platinum Group of the Periodic Table (Ru, Rh, Pd, Re, Os, Ir, and Pt) or gold. Titanium (Ti) is also especially suitable. The transducer may be further coated with a biocompatible layer such as Parylene or Parylene C.

Commonly assigned patent publication nos. US20020128647A1; US20020138074A1; US20030130657A1, and US20050107783A1; disclose additional variations of transducer assemblies and modes of securing such assemblies to the device. The entirety of each of which is incorporated by reference herein.

Moreover, variations of the inventive device include conducting members that comprise a series of wires, with one set of wires being coupled to respective poles of the transducer, and any number of additional sets of wires extending through the device. In addition, the sensing element 206 may have more than one sensing surface disposed along the portion of the sheath.

FIG. 2A also illustrates the far end of the sheath 202 as having the sensing element 206 offset and extending distally beyond the sheath opening 216. As discussed below, such a configuration permits insertion of the sensing element 206 and far end of the sheath 202 into tissue while spacing the sheath opening 216 proximally from the tissue. One such benefit of this spacing is that when used with a scope type device, the physician is able to observe the entry of the needle tip 240 into tissue while the sensing element is pressed against the tissue. Such a feature is not possible if the sheath opening is flush with the sensing element.

The degree to which the sensing clement 206 is offset and extends from the sheath 202 can vary depending on the particular application. For example, in certain variations, the sensing tip may be immediately distal to the far end of the sheath. In alternate variations, the sensing tip may extend as shown in the drawings. It may extend, for example, by 0.1 to 0.3 inches and more preferably about 0.20 to 0.25 inch. Such a construction is useful when the practitioner desires to view the sensing clement 206 at any orientation when the device extends from the endoscope.

Offsetting the sensing element 206 from the sheath opening can be accomplished any number of ways (as shown below). However, in the present variation, a wall 218 of the sheath 202 that defines the secondary lumen 214 extends beyond or distal to the sheath opening 216.

FIG. 2A also illustrates the sheath 202 as having a puncture-resistant or reinforcing member 220 extending at least partially along the sheath 202. The reinforcing member 220 can be any structure such as a braid, coil, plurality of rings, a sequence of rings or cylinders, or a slit tube having a greater hardness or stiffness than the sheath 202 itself. The reinforcing member can have a concentric, oval, or D-shape cross-section. The benefit of an oval or D-shaped reinforcing member is that it better utilizes the cross-sectional area of the main lumen. The reinforcing member serves to prevent the internal working lumen from being punctured by the needle, or from catching or otherwise hindering the needle as it is extended through the working lumen of the catheter. Although the reinforcing member 220 is shown as being within the main lumen 212, the reinforcing member can also be placed within a wall of the sheath 202. The reinforcing material can be comprised of any number of materials commonly used in catheters or medical devices. Such materials may include stainless steel, alloys, or Nitinol. The outer diameter of the reinforcing member may range from 0.020 to 0.050 inches and more preferably from 0.040 to 0.043 inches. The wire thickness may be from 0.001 to 0.010 inches and more preferably in the range of 0.002 to 0.004 inches. In the preferred embodiment, the reinforcing member is concentrically placed and is a coil. The coil has a closed pitch winding. The major and minor diameters of an oval or D-shaped reinforced member may range from 0.030 to 0.065 inches and 0.020 to 0.050 inches, respectively. The wire thickness and pitch can be of the same range as the concentric reinforcing member.

FIG. 2A illustrates an embodiment of the device 200 in which the needle tip 240 is sharp and curved such that a front portion 250 of the needle tip curves towards the axis of the needle body or the centerline of the needle tip 240. The curved needle tip 250 reduces the likelihood that the needle tip damages or penetrates the interior of the sheath 202 as the physician advances the device around a bend or actuates the needle relative to the sheath when the device is in a tortuous path. The device may need to make turns of 90 to 180 degrees, and frequently from 120 to 160 degrees. Failure to smoothly turn in a bronchoscope, airway or another lumen or penetration of the sheath by the advancing needle, may result in damage to the bronchoscope or worse, the patient.

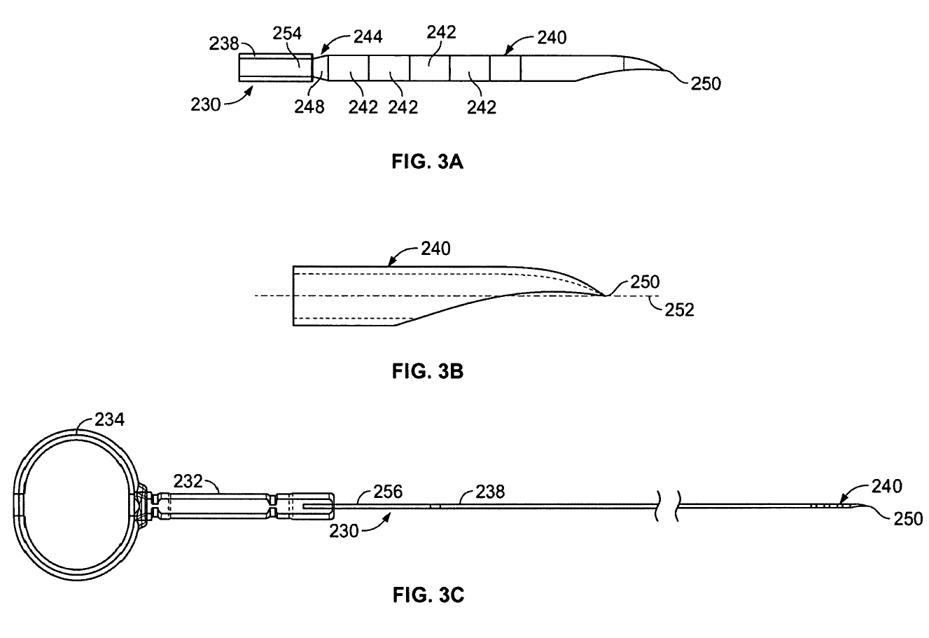

Various parameters and dimensions may be defined to characterize the needle tip 240 such as, but not limited to, the rigid length, outer diameter, inner diameter, bevel angle, and length of the bevel region as shown in FIG. 3B. The following dimensions are provided to be exemplary and are not intended to limit invention except where specifically incorporated into the appended claims. The rigid length (L) may range from 0.10 to 0.75 inches and preferably from 0.20 to 0.30 inches, and more preferably about 0.25 inches. The bevel angle (α) may range from 10 to 30 degrees and more preferably from 10 to 20 degrees. The bevel angle shown in FIG. 3B is about 15 degrees. The length of the bevel region (A) is equal to the total rigid length (L) less length (B). (B) can range from 0.1 to 0.5 inches and preferably from 0.1 to 0.2 inches. The length (B) shown in FIG. 3B is about 0.13 to 0.15 inches. Consequently, the length (A) of the bevel region is about 0.1 to 0.2 inches and about 0.10 to 0.15 inches. Additionally, the needle tip 250 curves or bends back towards a centerline axis 252. The outer diameter of the needle is in the range of 0.01 to 0.05 inches and more preferably from 0.030 to 0.035 inches.

FIG. 2B illustrates the device of FIG. 1 alter movement of the ring structure 234 against the handle portion 208 of the sheath 202. As shown towards a far end of the sheath 202, the needle assembly advances out of the sheath opening. The length of the needle assembly 230 can be selected to be greater than a length of the sheath 202 by a set distance. The length that the needle assembly advances from the sheath opening may also be controlled by selecting or adjusting the distance (X) between detents 400A, 400B shown in FIG. 3C. This sizing permits controlled advancement of the needle tip 240 out of the sheath opening and past the sensor clement 206 by a set amount. Such a feature can improve the safety of the device by preventing excessive advancement of the needle tip 240 from the sheath. As discussed above, in some variations it is desirable for the sheath handle 208 and needle hub 234 to be configured to facilitate single handed advancement of the needle assembly 230 out of the sheath 202.

FIG. 2C shows a magnified view of the far end of the sheath 202 with the needle assembly 202 advanced from the sheath opening 216. As noted above, in those applications where excessive advancement of the needle assembly 230 could cause unintended damage, the length of the needle assembly 230 shall be dependent upon the length of the sheath 202. Where the length of the needle assembly 230 is selected so that the needle tip 240 extends distally to the sensing clement 206 when the hub or ring 234 advances against the handle 208.

FIG. 2C also illustrates a variation of the needle tip 240 having features that assist the physician when using the device. As shown, the needle tip 240 can include a plurality of visually identifiable sections 242 as well as a depth limiting feature 244 to prevent excessive advancement of the needle tip 240 into tissue. As noted above, in view of the offset of the sheath opening 216 from the sensing element 206, the physician is able to maintain the sensing element 206 against tissue while observing the visually identifiable sections 242 to determine the depth of advancement of the needle tip 240 into tissue. The device sheath 216 and lumen 218 are preferably transparent or otherwise visually clear to allow the physician to observe the sections 242. In addition, the depth-limiting feature or transition section 244 provides feedback to the physician by requiring increased force to continue advancement of the needle tip 240. The visually identifiable sections 242 can each have a different color, or a combination of colors, the sections can have varying surfaces so that they reflect light in different manners (e.g., a rough surface, a polished surface, etc.). Also, variations of the needle assembly 230 can include visually identifiable sections 242 having different lines or other markings.

FIG. 3A shows another view of the distal end of the needle assembly 230. As shown, one example of a depth limiting feature is provided as a transition section 244 located between the needle tip 240 and an elongate member 238. The transition section 244 can be any configuration that provides interference with tissue to provide the physician with some tactile or sensory feedback that the transition section 244 has entered tissue. In the illustrated variation, the transition section 244 consists of a tapered section 248 of the needle tip 240 that abruptly joins an end of the elongate member 238. As shown, the needle tip 240 can be joined to the elongate member 238 via a shaft 254 or any other means. For example, the elongate member 238 and needle tip 240 could be fabricated from a single piece of material where the transition section is machined or cut into a desired location.

The tapered section 248 is smaller in diameter than an end of the elongate member 238. The joining of the two structures creates an irregular or discontinuous surface to provide the depth limiting feature. When used in tissue, as the needle tip 240 penetrates tissue, the curved tip 250 penetrates the tissue to conform to the outer diameter of the needle tip 240. The needle does not core or remove significant amounts of tissue. Once the needle 240 advances such that a portion of the tissue surrounds the transition section 244, the tissue recovers and conforms to the tapered section 248. Further advancement of the needle assembly 230 causes the tissue to engage the irregular or discontinuous transition to the distal end of the elongate member 238.

FIG. 3A illustrates a tapered section 248 smaller in diameter than the needle 240. However, another variation of the invention includes providing a transition section that is larger in diameter than that of the elongate member 238 and needle. Protrusions, wings, shoulders, or a step may provide tactile feedback and indicate the full extension of the needle or the relative depth of the needle into the tissue. The depth limiting structure may be located a distance ranging from 0.25 to 1.50 inches from the needle tip 250, and more preferably from 0.40 to 0.50 inches from the needle tip 250.

FIG. 3B shows a variation of a needle 240 as described above. As shown, the needle can include a beveled tip 250 similar to that of a lancet or septum penetrating needle. The curved tip 250 angles towards a centerline 252 of the needle 240. Variations of the device include a curved tip 250 that does not curve to the centerline or curves past the centerline. The curved tip reduces the chance that the needle tip 250 interferes or damages a wall of the sheath, and subsequently the bronchoscope or endoscope as the physician advances the needle. The needle can be constructed of various materials commonly used in similar medical applications including, but not limited to stainless steel, Nitinol, metal alloy, etc.

FIG. 3C shows a needle assembly 230 as described above. The needle tip 240 is joined to the hub 232 via an elongate member 238. In variations of the device used in the airways or other tortuous anatomy, the elongate member 238 is sufficiently flexible to navigate through tortuous anatomy while having column strength to penetrate tissue when the force is applied at the hub 232 or thumb-ring 234. In some variations, the elongate member 238 can include a reinforced section 256 adjacent to the hub 232. The reinforced section 256 can be designed with increased stiffness to prevent buckling or kinking of the elongate member 238. The elongate member 238 may be a hollow tubular member, or filled or solid.

The needle 240 is joined to the elongate member 238 by, for example, a press fit, welding, an outer sleeve, heat shrinking tube, wrap, or an adhesive. The needle preferably has a relatively short length so that it may turn corners ranging from 90-180 degrees. In applications in the lung, for example, it is not uncommon to make a turn greater than 120 degrees. One application that requires the physician to navigate the instrument through a sharp turn is when it is necessary to access a site in the upper lobe of*the lung. The length of the needle 240 in this embodiment preferably ranges as indicated above.

In addition, or as an alternative to the needle configurations shown above, the sheath can be combined with various aspiration needles, transbronchial aspiration needles, biopsy devices, other such commonly known devices where removal of tissue or other fluids is desired.

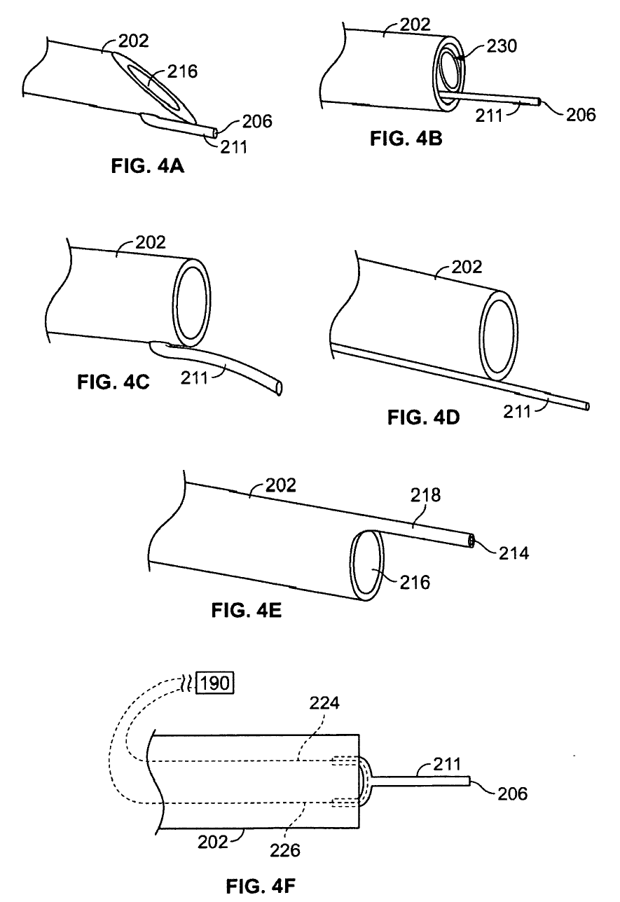

FIG. 4A shows a variation of a sensing element 206 having a segment 211 that extends from the tip of the sheath 202 and where the sheath opening 216 is tapered. The taper may be linear or gradually curved. As discussed herein, in most variations, the sensing element 206 is fixed and extends a distance beyond the sheath 202 so that the sensing element 206 can be pressed against tissue to scan for blood vessels or other structures. Once the physician locates an acceptable site, the physician advances the working device to perform a procedure at the site.

FIG. 4B illustrates another variation of a sensing element 206 extending from the far end of a sheath 202. In this variation, the sensing element 206 is located on an extension segment 211 that is affixed to the sheath 202. FIG. 4B also shows a working device 230 extending within the sheath lumen 202. The segment 211 may extend through the length of the sheath 202 or may be terminated near the far end of the sheath 202 with the conductive elements (e.g., wires) extending to the control system (not shown). In some variations, the segment 211 extends through the device but the portion extending from the far end of the catheter is stiff/has a sufficient column strength to probe tissue while a remainder of the segment has a lower stiffness/column strength to accommodate flexibility of the device. In any such constructions, the conductive element (or segment portion that extends in the device) docs not significantly reduce the ability to navigate the device through tortuous anatomy.

FIG. 4C illustrates another variation in which the sensing clement 206 is angled away from a central axis of the sheath 202 by using an offset segment 211. Such a feature is useful when trying to sense along a wall of a body passage since less articulation of the sheath 202 is required to contact the sensing element 206 against tissue.

FIG. 4D shows another variation of an offset sensing element 206. In this variation, the segment 211 may comprise a tube or similar member that extends along and externally to the length of the sheath 202.

FIG. 4E illustrates yet another variation, similar to that of FIG. 4A above, where the sheath opening 216 is not tapered. In this variation, the catheter 202 includes a second lumen 214 through which a sensing assembly (not shown) may be secured. As shown, a wall 218 of the sheath 202 extends beyond the sheath opening 216 to place the sensing element distal to the sheath opening 216.

FIG. 4F shows another variation in which a segment 211 of a sensing assembly is inserted into the far end of the sheath. The segment 211 may have connections 224, 226 for coupling to a control system 190 as described above. In such a variation, the sheath 202 may be a multi-lumen tube with one or more lumens reserved for the sensing assembly and connections. In addition, the location of the segment 211 can be offset as described above.

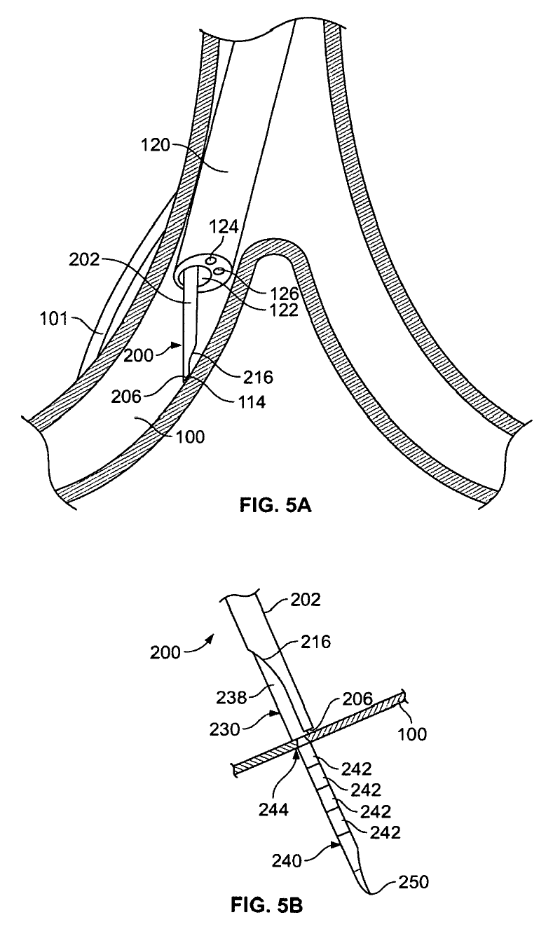

FIG. 5A illustrates an example of use of the devices described herein. Although the figures show a single variation, it is contemplated that any variation of the device may be substituted. In the illustrated example, the device creates an extra-anatomic passage in the airway wall tissue within a lung. However, it is understood that the device may be used in any part of the body and for any application. For example, variations of the device may be used during a biopsy procedure to scan for blood vessels, and remove a biopsy sample within the tissue piercing member.

FIG. 5A shows an access device 120 advanced into the airways 100 of a lung. The access device 120 may be a bronchoscope, endoscope, endotracheal tube with or without vision capability, or any type of delivery device. The access device 120 will have at least one lumen or working channel 122. In the illustrated version, access device 120 includes a light 124 and vision 126 capabilities. For example, location of the site may be accomplished visually, or with additional equipment such as a computer topography scanner to locate areas for treatment. In cases where the access device 120 is a bronchoscope or similar device, the access device 120 is equipped so that the surgeon may observe the intended target site 114. In some cases it may be desirable for non-invasive imaging of the procedure. In such cases, the access device 120 as well as the other devices discussed herein, may be configured for detection by the particular non-invasive imaging technique such as fluoroscopy, “real-time” computed tomography scanning, or other technique being used.

In certain minimally invasive applications and interventional procedures (for example, bronchoscopically accessing the upper lobe of the lung) turns of 90 degrees or more are required. Navigating, extending and retracting a needle is a challenge. In such cases, the needle tip design described above, in combination with a puncture resistant working lumen, serves to prevent damage to the catheter, bronchoscope, and patient.

FIG. 5A also illustrates advancement of a variation of the inventive device 200 through the channel 122 of the access device 120 towards the target site 114. The physician then uses the sensing element 206 located at a far end of the sheath 202 to inspect the target site 114 to determine whether a blood vessel 101 (or other structure) is adjacent to the site. If a blood vessel is detected at or near the site 114, then another target site may be selected.

The sensing tip 206 of the device 200 should be in contact with the airway wall to function properly. A physician will confirm contact with the airway wall using bronchoscopic vision by the displacement of the airway wall with light forward pressure on the bronchoscope and/or device. In variations using a Doppler Ultrasound mode of detection, movement of the device against the airway wall can potentially cause sounds that may not be distinguishable from sounds of blood vessels. Accordingly, a physician may hold the device still, and listen for a period (e.g., 2-3 seconds) to confirm whether a vessel is the cause of the sounds. Stopping and listening for the period is also required in order to confirm the absence of vessels. In one technique, the physician sweeps the tip of the sheath 202 across the target site 114. The scanning can include circumferentially and axially scanning the areas surrounding the target site. The physician will frequently stop the device to listen for the sounds associated with vessels (i.e. if a blood vessel is present the user will hear a pulsing sound or a swishing sound indicating blood flow).

As shown in FIG. 5B, once the physician determines that the site is free of any blood vessel, the physician can hold the device 200 at the vessel free location, maintain light pressure against the airway wall and then actuate the handle to expose the needle 240 and make a passage in the airway wall. In those variations where the needle 240 includes visual markers 242, the physician advances the needle tip 240 until the preferred indicator mark rests in the airway wall (e.g., any number of colors or shades may be used where the color is dictated by a shade that is easily distinguishable under bronchoscopic observation). Additionally, a radio opaque mark may be added to one or more of the segments to indicate the depth under X-ray or fluoroscopy. As an additional measure of safety, because the curved tip 250 does not core tissue, the tissue conforms to the surface of the needle tip 240 and then conforms to the transition region 244. The discontinuous or irregular surface of the transition region 244 acts as a depth-limiting feature as shown. As an added measure of safety, the physician can maintain visualization throughout this process, ensuring that the sensing tip 206 remains in the same location throughout the passage-making process and that the needle 240 penetrates to the selected visual mark 242. Such safety considerations minimize the chance that the device creates a passage of uncontrolled depth that could result in serious harm to the patient, such as excessive bleeding or a pneumothorax.

As noted above, the needle 240 can advance into the target site without removing the sensing element 206 from the tissue. Accordingly, variations of the device require sufficient stiffness so that the tissue may be adequately probed without collapse of the sensing element 206 or segment carrying the clement. As described above, the system 150 provides the physician with audio or visual signals so that the physician can determine whether it is sufficiently safe to make an opening in the tissue. The physician may rotate, probe, circulate, traverse, or otherwise move the tip 206 in the area to reach a sufficient degree of confidence that the location or site is blood vessel free.

A further variation of the invention may include configuring the transducer assembly and/or controller to have different levels of sensitivity. For example, a first level of sensitivity may be used to scan the surface of tissue. Then, after creation of the opening, the second level of sensitivity may be triggered. Such a feature acknowledges that scanning of tissue on, for example, the airway wall may require a different sensitivity than when scanning tissue within the parenchyma of the lung.

It should be noted that the invention includes kits containing the inventive device with any one or more of the following components, a Doppler ultrasound controller, a conduit delivery catheter incorporating a conduit thereon, as described in one or more of the applications listed above, and a bronchoscope/endoscope.

In the above explanation of Figures similar numerals may represent similar features for the different variations of the invention.

The invention herein is described by examples and a desired way of practicing the invention is described. However, the invention as claimed herein is not limited to that specific description in any manner. Equivalence to the description as hereinafter claimed is considered to be within the scope of protection of this patent.

The devices of the present invention are configured to locate a target site for creation of a collateral channel in the tissue and to create an opening in tissue. As discussed above, a benefit of this combination feature is that a single device is able to select a target location and then create an opening without having been moved. Although the device is discussed as being primarily used in the lungs to create a collateral channel, the device is not limited as such and it is contemplated that the invention has utility in other areas as well, specifically in applications in which blood vessels or other structures must be avoided while cutting or removing tissue (one such example is tumor removal) or in Transbronchial Needle Aspiration or Transbronchial Needle Biopsy.

The above illustrations are examples of the invention described herein. It is contemplated that combinations of aspects of specific embodiments/variations or combinations of the specific embodiments/variations themselves are within the scope of this disclosure.

We claim:

1. A medical device for sensing structures beneath tissue and penetrating tissue, the device comprising:
an elongate sheath having a near portion, a far portion, and a handle located on the near portion, the elongate sheath including at least one lumen extending through the handle to the far portion and exiting at a sheath opening, the elongate sheath being sufficiently flexible to navigate through tortuous anatomy, where the elongate sheath comprises an inner puncture-resistant reinforcing member;
a sensing element spaced distally from the sheath opening and positioned such that an active surface of the sensing element is able to scan in a distal direction to inspect tissue distal to a front tip of the elongate sheath while leaving the sheath opening proximally spaced from tissue, where the sensing element is coupleable to a sensing monitor;
an elongate body member having a hub located at a proximal end, and a needle tip located at a distal end, the body member and needle tip being slidably located through the sheath lumen, where a length of the body member is greater than a length of the elongate sheath such that when the hub advances against the handle, the needle tip extends distally to the sensing element and wherein said reinforcing member and needle tip cooperate with one another to ensure that the needle tip may be extended out of the sheath opening without engaging or damaging the wall of the sheath when the elongate sheath is extended through a bend of at least 120 degrees.

2. The medical device of claim 1, where the needle tip comprises a curved tip and where a front portion of the curved tip curves towards a centerline of the needle.

3. The medical device of claim 1, where the handle comprises at least one annular groove and the hub comprises a ring, such that the body member can be advanced using a single hand.

4. The medical device of claim 1, where the sheath member comprises a second lumen having a wall that extends distally beyond the sheath opening, and where the sensing element is located within the second lumen.

5. The medical device of claim 1, where a transition section between the distal end of the elongate body and the needle tip comprises a reduced diameter section distal to an increased diameter section causing the transition section to have an irregular surface that creates tactile feedback to a user upon insertion into tissue.

6. The medical device of claim 1, where the needle tip comprises a length of between 0.1 and 0.3 inches.

7. The medical device of claim 1, where the needle tip includes a plurality of visually colored sections.

8. The medical device of claim 1, where the needle tip and elongate body member comprises a configuration selected from the group consisting of an aspiration needle, a transbronchial aspiration needle, and a biopsy coring device.

9. The medical device of claim 1, where the sensing element comprises a transducer assembly.

10. The medical device of claim 1, where the reinforcing member is a coil having a diameter between 0.035 and 0.045 inches and a closed pitch.

11. The medical device of claim 1, where the reinforcing member comprises a structure selected from a braid, a coil, a plurality of rings, and a slit tube.

12. A medical device for sensing structures beneath tissue and penetrating tissue at a remote site in tortuous anatomy, the device comprising:
an elongate sheath having a near portion, a far portion, and a handle located on the near portion, the elongate sheath including at least one lumen extending through the handle to the far portion and exiting at a sheath opening, the elongate sheath having a reinforcing member extending at least partially therethrough and being sufficiently flexible and long to navigate through tortuous anatomy;
a sensing element being coupleable to a sensing monitor;
an elongate body member having a hub located at a proximal end, and a needle tip located at a distal end, the body member and needle tip being slidably located through the sheath lumen, where a length of the body member is greater than a length of the elongate sheath such that when the hub advances against the handle, the needle tip extends distally from the sheath opening, where the elongate body member has a column strength to allow the needle tip to puncture tissue when the hub advances against the handle: and
where the needle tip comprises a curved tip and where a front portion of the curved tip curves towards a centerline of the needle tip thereby minimizing the front portion of the curved tip from interfering with an interior wall of the sheath lumen.

13. The medical device of claim 12, where a transition section between the distal end of the elongate body and the needle tip is discontinuous such that advancement of the needle tip and the adjacent surface into tissue creates tactile feedback to a user.

14. The medical device of claim 13, where the transition section comprises a reduced diameter section distal to an increased diameter section.

15. The medical device of claim 12, where the sensing element is spaced distally from the sheath opening and positioned such that an active surface of the sensing element is able to scan in a distal direction to inspect tissue distal to a front tip of the elongate sheath while leaving the sheath opening proximally spaced from tissue.

16. The medical device of claim 12, where the sheath member comprises a second lumen having a wall that extends distally beyond the sheath opening, and where the sensing element is located within the second lumen.

17. The medical device of claim 12, where the handle comprises at least one annular groove and the hub comprises a ring, such that the body member can be advanced using a single hand.

18. The medical device of claim 12, where proximal of the needle tip includes a plurality of visually colored sections.

19. The medical device of claim 12, where the sensing element comprises a transducer assembly.

20. The medical device of claim 12, where the elongate sheath member comprises a reinforcing member extending at least partially therethrough.

21. The medical device of claim 12, where the reinforcing member comprises a structure selected from a braid, a coil, a plurality of rings, and a slit tube.

22. A method for surgically creating a channel in a wall of an airway within the lung, comprising:
providing a catheter, said catheter comprising a distal section and said distal section comprising an Ultrasound Doppler transducer and a sharp, advanceable, tissue penetrating member;
advancing the catheter through a natural respiratory opening and into a first airway,
advancing the catheter across a bend in the first airway of at least 90 degrees, and to a first location along the first airway wall and distal to said bend;
sensing in the vicinity of said first location for the absence of blood vessels with said Doppler transducer;
extending the needle from the distal section of the catheter to create a first channel through said first airway wall without breaching or damaging the internal lumen of the catheter wherein the needle comprises a rigid length of between 0.1 to 0.5 inches and the internal lumen of the catheter comprises a reinforcing member.

23. The method of claim 22 wherein the reinforcing member is a coil.

24. The method of claim 22 wherein said bend in the first airway is at least 120 degrees.

25. The method of claim 22 wherein said first location is in said upper lobe of said lung.

26. The method of claim 22 comprising navigating said catheter to a second location in a second airway of said lung.

27. The method of claim 26 comprising creating between 2 to 6 channels in 2 to 6 different airway walls.
